(19) United States
(12) Patent Application Publication
(10) Pub. No.: US 2008/0190527 A1
(43) Pub. Date: Aug. 14, 2008

(54) Disposable cover for shopping device handles

(76) Inventors: Renee D. Guercia, East Norwich, NY (US); Suzette A. Guercia, East Norwich, NY (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 11/704,011

(22) Filed: Feb. 8, 2007

(51) Int. Cl. A61B 19/02

(52) U.S. Cl. 150/155

(57) ABSTRACT

A single-use disposable sanitary cover temporarily houses a handle of a shopping device or hospital cart. The disposable sanitary cover includes a flexible cylindrical sleeve having an elongated slit, and stabilizing means integrally associated therewith. The stabilizing means mitigates movement of cylindrical sleeve along the hospital cart's or shopping device's handle. The handle of the shopping device or hospital cart is received through the elongated slit and is housed within the cylindrical sleeve in order to mitigate the viral and germ contact between the handle of the shopping device or hospital cart and the shopper's or hospital worker's hands. Preferably, the disposable sanitary cover includes a tab member for removing the disposable sanitary cover from the handle of the shopping cart.

### BACKGROUND OF THE INVENTION

1. Field of the Invention

The present invention relates to a disposable cover for shopping device handles; and more particularly, to a disposable sanitary cover that is adapted for single-use and is clipped onto a shopping device or hospital cart handle to prevent germs resident on the handle from contacting a shopper's or hospital worker's hands.

2. Description of the Prior Art

The spread of viruses and germs via the contact interface between a person's hands and a shopping cart handle or the like, concerns many consumers. Shopping carts tend to carry germs on their handles, sine a single cart can be utilized by dozens of individuals each day. Just positioning a child in a shopping cart seat will immediately place the child, as well as the user, in grave danger of contacting harmful, unhealthy germs. Attempts have been made to provide measures to avoid or mitigate contact with these germs. However, many of these devices fail to provide a cover device that is readily clipped onto the handle of a shopping cart or basket and is equipped with stability means that prevent the clip from moving lengthwise and circumferentially along the handle.

Conventional measures for mitigating contact with germs have included shopping cart handle and cover assemblies in which a releasable handle receives a removable cover. For example, U.S. Pat. No. 2,803,849 to Peters discloses a sanitary shopping cart handle pivotally mounted on one side of a shopping carrier. The handle pivots open, enabling insertion of tubular paper cover, which encompasses the handle. Unfortunately, these shopping cart handle and cover assemblies are limited to shopping carts having specific handle constructions. Sanitary shopping cart handles of this type require modification of the traditional shopping cart, which generally do not have handles capable of pivoting open for inserted of a tubular cover. The cover is not susceptible of use with carts extant in a variety of different stores or shopping markets.

Other handle cover apparatus provides directory and/or clip board combinations. For example, U.S. Pat. No. 2,888,761 to Miller discloses a directory and clip board combination appointed to be attached to a shopping cart handle. The directory and clip board device includes a panel for securing papers, cards, or the like, with a cut-away so that a user's hands can access the shopping cart handle. U.S. Pat. No. 4,512,504 to Owlett discloses a shopping organizer appointed for attachment to a handle of a shopping cart. These cover directory/organizer combinations generally fail to provide a sanitary surface to prevent the spread of germs. Their objective is to provide a directory or organizer that can be clipped onto the handle of a cart, and they are not adapted to prevent contact with germs on the carts' handles. As a result, the user is exposed to a plethora of germs, which are left on the handle by previous cart users.

Still other combinations provide a sanitary cover for infants while equipping the infant with an entertaining environment. For example, U.S. Pat. No. 6,491,996 to Digangi discloses a sanitary cover for use on a shopping cart, infant seat or similar child seating device, wherein the cover is appointed with tabs which carry fasteners to secure the cover in place. Perforated areas are also provided. These perforated areas can be removed or punched out to allow the child's legs to extend through the cover and through the openings. U.S. Patent Application Publication No. 2004/0189066 to Beaty discloses a sanitary, portable and disposable cover for shopping cart handles and surrounding cart framework. A cover is adhered and stretched taut across a space between a horizontal bar and a restraint railing of a cart. The cover provides an activity center with images and a tray for holding snacks or toys. These infant covering combinations are not concerned solely with covering of the handle region. Instead, the covering includes a region adjacent to the area where the infant will be sitting. Accordingly, these covering combinations do not provide a simple cover for that is readily placed upon the handle region to shield the user against infectious germs.

Various shopping cart handle covers have been suggested in which the cover is constructed with a generally tubular body and provided with a slot or slit for temporary insertion onto a shopping cart handle during shopping. For example, U.S. Pat. No. 5,215,319 to Farris discloses a sanitary handle for shopping carts providing a disposable sanitary cover made from a flexible tubular member having an elongated tubular body that has a slot cut longitudinally the entire length of the elongated tubular body. One end of the slot has a notch which is pressed so that the cover can be inserted over the handle of a shopping cart. U.S. Pat. No. 6,065,764 to Moseley discloses a sanitary cover that removably engages a shopping cart handle comprised of a sheet of non-toxic, hypoallergenic durable, flexible material, defining a cylindrical cavity having a slit. U.S. Design Pat. No. Des. 370,110 to Beam discloses an ornamental design for a shopping cart handle cover showing a tubular cover with a slit. U.S. Design Pat. No. Des. 521,207 to Anderson et al. discloses an ornamental design for a sanitary article for shopping cart handles showing a tubular construction with a lip running lengthwise thereof juxtaposed to a slit. U.S. Patent Application Publication No. 2002/0092132 to Kessler discloses a protective sleeve for shopping cart handles, wherein the sleeve is slit to form a tube with a gap, and a plurality of notches for receiving vertical wires of carts extend from edges proximate to the slit and away from the gap. Intended disadvantages are shared by these constructs. Such disadvantages include unwanted shifting of the tubular body to and fro horizontally along the shopping cart handle, as well as rotating of the cover. Due to the shifting of the covering, gaps or openings can occur between the handle and the cover. The user's hand eventually comes into contact with the surface of the handle, and, consequently, the germs thereon, thereby negating use of the sanitary cover.

Assorted shopping cart handle covers are constructed to cover the entire handle as well as lateral portions thereof. For example, U.S. Pat. Nos. 5,427,392, 5,429,377 and 5,820,142 to Duer disclose sanitary protective covers for shopping cart use, including a main portion between ends having two cowls or flaps that extend over the side portions of the handle of the shopping cart. These handle coverings encompass the entire handle of the shopping cart as well as the side bars thereof. Where carts have varying handle lengths, the devices of these types of patents may not be able to be received on the handle.

Reusable protective coverings have been provided. These include constructs generally having a flat sheet that is wrapped around the handle and typically held in place via VELCRO (trademarked). U.S. Pat. No. 5,722,672 to Frederick discloses a reusable protective cover for shopping cart handle/rented strollers by providing a T-shaped handle cover having a horizontal section appointed for covering the horizontal handle and a vertical section for wrapping around a vertical bar of the cart's seat basket frame. U.S. Pat. No. 6,543,794 to Tyree discloses a shopping cart sanitary handle cover comprising a first semi-flexible sheet of vinyl and a second sheet of fabric superimposed thereon and heat bonded thereto with corresponding lateral edges. The sheets are wrapped about a shopping cart handle bar and secured with hook and loop fasteners to maintain the cover on the handle bar. U.S. Pat. No. 6,832,766 to Stokes discloses a protective covering for a handle comprised of a front portion, objects, and attachment portions comprised of hooks or loops, i.e. a first VELCRO (trademarked) hook and loop fastener portion. The protective covering is wrapped around and attachable to a handle. U.S. Pat. No. Des. 328,812 to Pritchett discloses an ornamental design for a handle cover for a shopping cart wherein the cover is flat and is wrapped around the handle and secured in place. U.S. Patent Application Publication No. 2002/0000700 to Alexsen et al. discloses a disposable shopping cart sheath having a generally triangular shape defining that is appointed to be wrapped around the handle of a shopping cart. Further reusable protective coverings have been provided that involve constructs having a tubular shape and means for placing the tube onto the cart's handle and securing same thereto. For instance, U.S. Pat. No. 6,817,066 to Williams et al. discloses a sanitary grip apparatus that may be installed on a shopping cart handle which has a tube construction with a slit and integrated pull-tabs to provide a means to install the grip without touching the surface of the shopping cart handle.

These aforementioned reusable protective coverings are not discarded after use, but are instead saved and carried for later application. Over time, germ buildup on the reusable coverings themselves can occur. Moreover, many of these protective coverings do not provide a tube-like construction that is readily attached to a cart handle. Instead, they provide a flat sheet that is wrapped around and secured to the handle. Even where the reusable coverings can accommodate a tubular shape, the coverings do not provide portions which ensure that the covering does not shift during use while also providing the ability to readily attach and detach the covering effortlessly.

Length adjustable handle coverings have been provided. Examples of such constructs include the following: U.S. Pat. No. 6,981,707 to Dandy discloses an adjustable portable protective cover for covering a shopping cart handle. Three elongated resilient tubular member are telescoped together so that the protective cover can be collapsed to allow the user to conveniently carry the protective cover to and from the store where the shopping carts are utilized. U.S. Design Pat. No. Des. 298,077 to Goodwin discloses an ornamental design for an adjustable snap-on hand grip for a shopping cart or the like. A telescoping portion of the construct enables the cover to be adjusted. Generally, length adjustment is achieved via telescoping portions that collapse together to form a more condense body for carrying. Comfort during use of the handle may be compromised if the user's hands or fingers come into contact with the overlapping sections. This can potentially lead to pinching of the users palm or fingers.

Additionally, coverings having multiple layers have been provided wherein a topmost layer is used and discarded to expose another layer therebeneath. For example, U.S. Pat. No. 6,869,085 to Pettigrew et al. discloses a disposable shopping cart handle sanitary cover having multiple layers stacked one on top of another. A bottommost layer is attached to a grip on the cart. The topmost layer of the sanitary cover is removable for exposing another layer there-beneath, thereby providing a sanitary surface adapted to be gripped by a user. U.S. Patent Application Publication No. 2005/0206104 and Foreign Publication No. WO 2003/045756 to Pettigrew et al. discloses a disposable shopping cart handle sanitary cover having multiple layers stacked one on top of another. A bottommost layer is attached to a grip of the cart's handle so that a topmost layer of the cover is removable to expose another layer there-beneath. Moreover, U.S. Patent Application Publication No. 2004/0021279 to Sobo et al. discloses a sanitary cover for shopping cart handles. A sheet is adapted to wrap approximately twice around the shopping cart handle and securely adhere thereto by way of static adhesion between the surfaces of the sheet. The assembly may be provided as multiple or stacked perforated layers. These multi-layered devices are attached to a cart's handle and remain there, presumably until the final layer is used and removed. Potential contamination may result as one user removes his or her used layer, exposing the new layer, which then may come into contact with a person's hands before another person uses the layer, resulting in germ passage.

Notwithstanding the efforts of prior art workers to construct a ready to use shopping cart handle covering that prevents or mitigates germ contact, there remains a need in the art for a disposable sanitary cover adapted to temporarily house a handle of a shopping device, and which is characterized by the presence of an elongated tubular construction with a slit therein, so that the covering is adapted to be clipped onto a shopping cart handle. Specifically, there is a need in the art for a disposable handle covering having a tubular construction provided with a slit therein, and additionally provided with stabilization means to prevent shifting and rotation thereof during use. Further, there remains a need in the art for a handle covering that is disposable after a single use, and which thereby avoids contamination through reuse and during placement of another's hands thereon.

### SUMMARY OF THE INVENTION

The present invention provides a disposable sanitary cover for shopping cart handles, as well as a method for use thereof. The disposable sanitary cover for shopping cart handles is in essence a handle covering having an elongated tubular sleeve construction. The sleeve has a slit therein for readily clipping the cover onto the shopping device's handle. It is further provided with stabilizing means. This stabilizing means prevents movement of the elongated cylindrical sleeve lengthwise along the shopping device's handle, while at the same time preventing rotational movement along the circumference of the handle. Prevention of lengthwise and circumferential movement along the shopping device's handle averts unwanted exposure to contaminated portions of the handle and thwarts damage to the cover during use as the shopper's hands are repeatedly placed on and off the shopping device's handle and, as a consequence, the disposable sanitary cover.

The disposable sanitary cover for temporarily housing a handle of a shopping device and mitigating virus and germ contact on a shopper's hands includes a cylindrical sleeve. The cylindrical sleeve comprises a first end, second end, and a central portion. It is composed of a flexible material. An elongated slit in the sleeve forms a first rim and a second rim within the cylindrical sleeve. The disposable sanitary cover additionally includes a stabilizing means integrally constructed within the cylindrical sleeve. A stabilizing means is provided to mitigate movement of the cylindrical sleeve along the shopping device's handle. The handle of the shopping device is received through the elongated slit and is housed within the cylindrical sleeve in order to mitigate virus and germ contact between the handle of the shopping device and the shopper's hands. Preferably, the disposable sanitary cover includes a tab member for removing the disposable sanitary cover from the handle of the shopping cart.

The method of using a disposable sanitary cover for temporarily housing a handle of a shopping device for mitigating viral and germ contact on a shopper's hands includes the steps of removing a cylindrical sleeve from a dispensing device. The cylindrical sleeve comprises a first end, second end, a central portion, and an elongated slit forming a first rim and a second rim. The cylindrical sleeve is composed of a flexible material and additionally comprises a stabilizing means integrally associated therewith. A stabilizing means is provided to mitigate movement of the cylindrical sleeve along the shopping device's handle. Next, the method provides for aligning of the handle of the shopping device with the elongated slit and the first and second rims of the cylindrical sleeve. Lastly, the cylindrical sleeve is pressed downward so that the handle of the shopping device traverses the first and second rim and the elongated slit, so that the handle is inserted and temporarily housed within the cylindrical sleeve. The handle of the shopping device is received through the elongated slit and is housed within the cylindrical sleeve in order to mitigate viral and germ contact between the handle of the shopping device and the shopper's hands.

In another embodiment, the method of using a disposable sanitary cover for temporarily housing a handle of a shopping device for mitigating viral and germ contact on a shopper's hands comprises a first step of removing a cylindrical sleeve from a dispensing device. The cylindrical sleeve comprises a first end, second end, and a central portion, wherein the cylindrical sleeve further comprises an elongated slit forming a first rim and a second rim and the sleeve is composed of a flexible material. The cylindrical sleeve additionally comprises a stabilizing means integrally associated therewith. The stabilizing means mitigates movement of the cylindrical sleeve along the shopping device's handle. In the second step, the method requires aligning the handle of the shopping device with the elongated slit and the first and second rims of the cylindrical sleeve, wherein the first rim of the cylindrical sleeve further comprises a first flap appointed with a fastening strip. The cylindrical sleeve further comprises a tab assembly with an elongated strip traversing thereupon. In the third step, the cylindrical sleeve is pressed downward so that the handle of the shopping device traverses the first and second rim and the elongated slit, and is inserted and temporarily housed within the cylindrical sleeve. Next, the first flap of the first rim is folded over the second rim of the cylindrical sleeve and is attached to the second rim by the fastening strip. The attachment is such that the handle of the shopping device is completely and temporarily housed within the cylindrical sleeve. The shopping device is then used for a single shopping outing, after which the cylindrical sleeve is removed from the handle of the shopping cart by lifting the tab assembly of the cylindrical sleeve and puling the tab assembly and elongated strip so that the elongated strip traverses the cylindrical sleeve, tearing same and forming a release gap. The cylindrical sleeve is then removed from the handle of the shopping device by way of the release gap, and is discarded.

### BRIEF DESCRIPTION OF THE DRAWINGS

The invention will be more fully understood and further advantages will become apparent when reference is had to the following detailed description of the preferred embodiments of the invention and the accompanying drawings, in which:

FIG. 1a illustrates a front view of an embodiment of the disposable sanitary cover for temporarily housing a handle of a shopping device or hospital cart wherein the stabilizing means comprises tapered portions;

FIG. 1b illustrates a back view of the embodiment of the disposable sanitary cover for temporarily housing a handle of a shopping device shown in FIG. 1a;

FIG. 2a illustrates a front view of another embodiment of the disposable sanitary cover for temporarily housing a handle of a shopping device or hospital cart wherein the stabilizing means comprises a friction layer;

FIG. 2b illustrates a back view of the embodiment of the disposable sanitary cover for temporarily housing a handle of the shopping device or hospital cart shown in FIG. 2a;

FIG. 3a illustrates a front view of another embodiment of the disposable sanitary cover for temporarily housing a handle of a shopping device or hospital cart, wherein the cylindrical sleeve further comprises a first flap located on the first rim;

FIG. 3b illustrates a back view of the embodiment of the disposable sanitary cover for temporarily housing a handle of the shopping device or hospital cart shown in FIG. 3a; and

FIG. 4 illustrates a front view of another embodiment of the disposable sanitary cover for temporarily housing a handle of a shopping device or hospital cart wherein the stabilizing means comprises band portions.

### DETAILED DESCRIPTION OF THE INVENTION

The present invention provides a disposable sanitary cover for temporarily housing a handle of a shopping device, hospital cart or the like. It also provides a method for using the sanitary cover. The disposable sanitary cover comprises a cylindrical sleeve with an elongated slit therein, and a stabilizing means. Advantageously, the disposable sanitary cover is readily and effortlessly clipped onto the handle of the shopping cart, hospital cart or shopping basket. In turn, the disposable sanitary cover is readily removed from the handle after it has been used. A stabilizing means is provided to mitigate lengthwise and circumferential movement of the disposable cover on the handle of the shopping device or hospital cart. Prevention of lengthwise and circumferential movement (rotational movement) along the handle protects the user's hands from contacting exposed areas of the handle that can result from otherwise unwanted shifting of the cover.

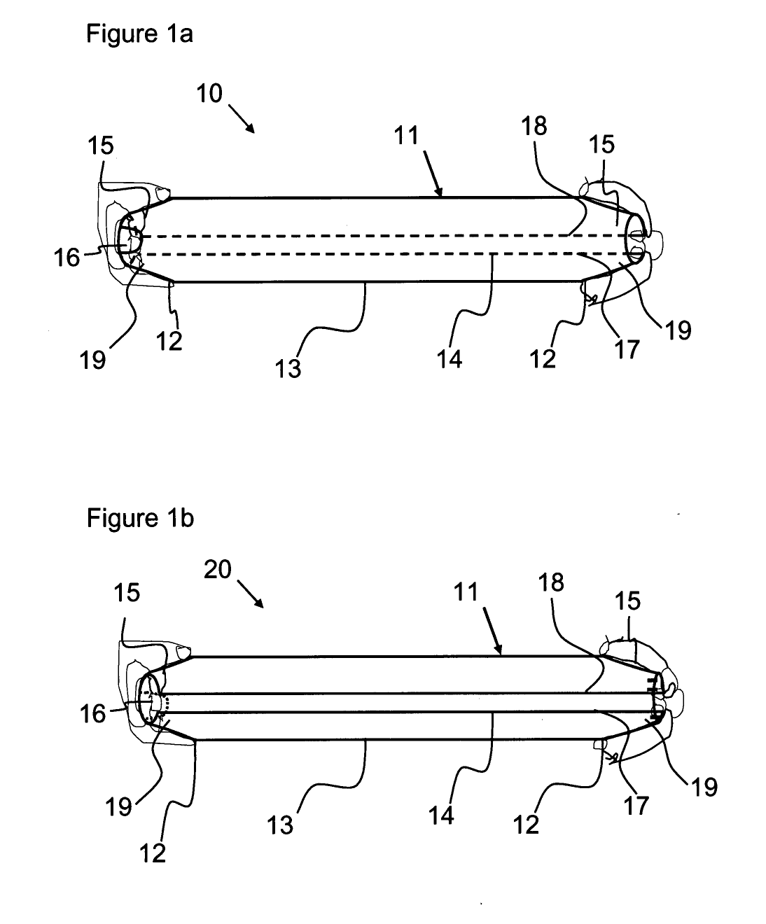

FIGS. 1a and 1b illustrate views of an embodiment of the disposable sanitary cover for shopping device handles wherein the stabilizing means comprises tapered portions located on the first and second ends of the cylindrical sleeve. Particularly, FIG. 1a illustrates a front view shown generally at 10, while FIG. 1b illustrates a back view shown generally at 20. Disposable sanitary cover 10, 20 is appointed to be received on a handle of an array of devices, including typical shopping carts pushed by a shopper and/or shopping baskets carried by a shopper, or hospital carts and the like. In this embodiment, the disposable sanitary cover 10, 20 includes a cylindrical sleeve 11 having first and second ends 12 and a central portion 13. Cylindrical sleeve 11 is composed of a flexible material. Preferably, cylindrical sleeve 11 is composed of a polymeric material, such as a thin plastic material, but should be recyclable in nature. Most preferably, cylindrical sleeve 11 is composed of a biodegradable coated cardboard or fiber, so that disposable sanitary cover 10, 20 is lightweight, easy to use, and environmentally friendly. Cylindrical sleeve 11 may comprise hand grip indentations located near first and second ends 12 thereof to facilitate grasping of the cover 10, 20 and provide comfort. Preferably, disposable sanitary cover 10, 20 shall be appointed to be dispensed in an interior entry way of a supermarket, next to or near shopping carts. Alternatively, disposable sanitary cover 10, 20 may be provided in a sealed packaging appointed to house disposable sanitary cover 10, 20 prior to use in a clean environment. Disposable sanitary cover 10, 20 can be designed with a logo of a store, or can be used as an advertising tool by any large company, by way of same on the surface of cylindrical sleeve 11. Disposable sanitary cover 10, 20 is preferably disposed of after a single usage.

A tab member 16 is preferably integrated within cylindrical sleeve 11 so that disposable sanitary cover 10, 20 can be removed from the handle of the shopping cart without the user having to touch the shopping cart handle. That is to say, tab member 16 is simply flipped-up and pulled upward upon so that the disposable sanitary cover 10, 20 is removed from the shopping device's handle. If not for this tab member 16, the user would generally have to grasp a portion of the shopping device, likely an exposed area of the handle of proximate area thereof, with one hand and grasp the disposable sanitary cover 10, 20 with the other and pull upward to effect the removal thereof. Unfortunately, such removal will cause contact of the user's hand with that portion of the shopping device, negating the intended purpose of the cover 10, 20, to prevent germ contact. Tab member 16 provides the ability to remove the cover 10, 20 without exposing the user to the germs on the shopping device.

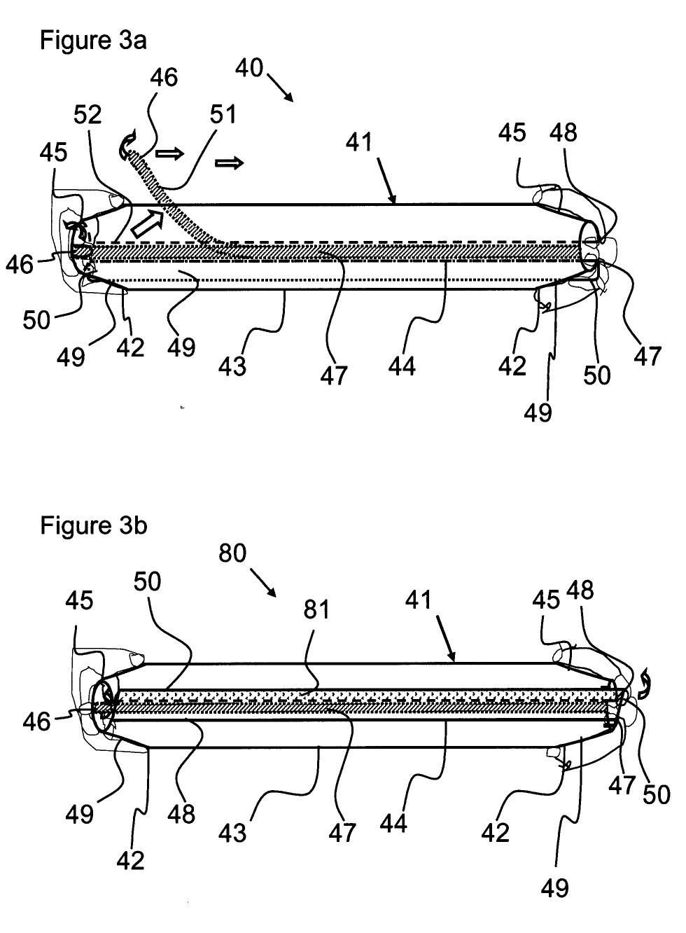

Cylindrical sleeve 11 further comprises an elongated slit 14. Slit 14 extends from each of the first and second ends 12 of cylindrical sleeve 11 forming a first rim 17 and a second rim 18 within cylindrical sleeve 11. Slit 14 is herein shown so that first and second rims, 17 and 18, of cylindrical sleeve 11 substantially abut one another. Alternatively, slit 14 can be arranged so that first and second rims, 17 and 18, of cylindrical sleeve 11 substantially overlap, thereby preventing any gaps, which could cause exposure to the handle of the shopping device when cylindrical sleeve 11 is placed thereon (preferably by way of the embodiment shown in FIGS. 3a and 3b discussed hereinbelow). A handle of a shopping cart appointed to be pushed by a shopper or a handle of a shopping basket appointed to be carried by a shopper is received through elongated slit 14 so that the handle is housed within flexible cylindrical sleeve 11 so that the passage of germs from the shopping cart's (or basket's) handle to a shopper's hands is mitigated.

Disposable sanitary cover 10, 20 further comprises a stabilizing means 15 integrally constructed therein so as to diminish movement of cylindrical sleeve 11 lengthwise and circumferentially on the handle of the shopping device or hospital cart. Lengthwise movement of disposable sanitary cover 10, 20 back and forth on the handle of the shopping cart yields both unwanted exposure of portions of the handle while at the same time tends to cause damage to the cover. Rotational movement around the circumference of the handle yields unwanted exposure to the elongated slit 14. It can potentially cause discomfort to the shopper's hands and may damage cover 10, 20. Stabilization means 15 is provided to avoid such lengthwise and circumferential movement. In this embodiment, stabilization means 15 comprises tapered portions 19 located on first and second ends 12 of cylindrical sleeve 11. Tapered portions 19 have smaller radii than central portion 13 and first and second ends 12 so that tapered portions 19 tightly embrace the hospital cart or shopping device's handle. In this manner, tapered portions 19 tightly grasp the handle of the hospital cart, shopping cart or basket in order to mitigate or prevent lengthwise shifting and circumferential rotation of cylindrical sleeve 11.

Prevention of the cylindrical sleeve 11 from rotating and/or moving to and from while placed on the handle of the shopping cart, hospital cart or basket has advantageous in that same mitigates unwanted exposure of the user's hands to handle areas that are sought to be covered. That is to say, that without the ability to prevent rotation of the cylindrical sleeve 11; the sleeve would readily rotate so that the slit 14 could expose the user to the handle of the cart at the point of the slit 14. The ability to prevent movement to and from of the cylindrical sleeve 11 mitigates the risk of the hands of the user coming into contact with the handle. Also, the stabilization means mitigates the risk that the cylindrical sleeve 11 will fall off of the handle of the basket or cart after repeated hand activity of the user.

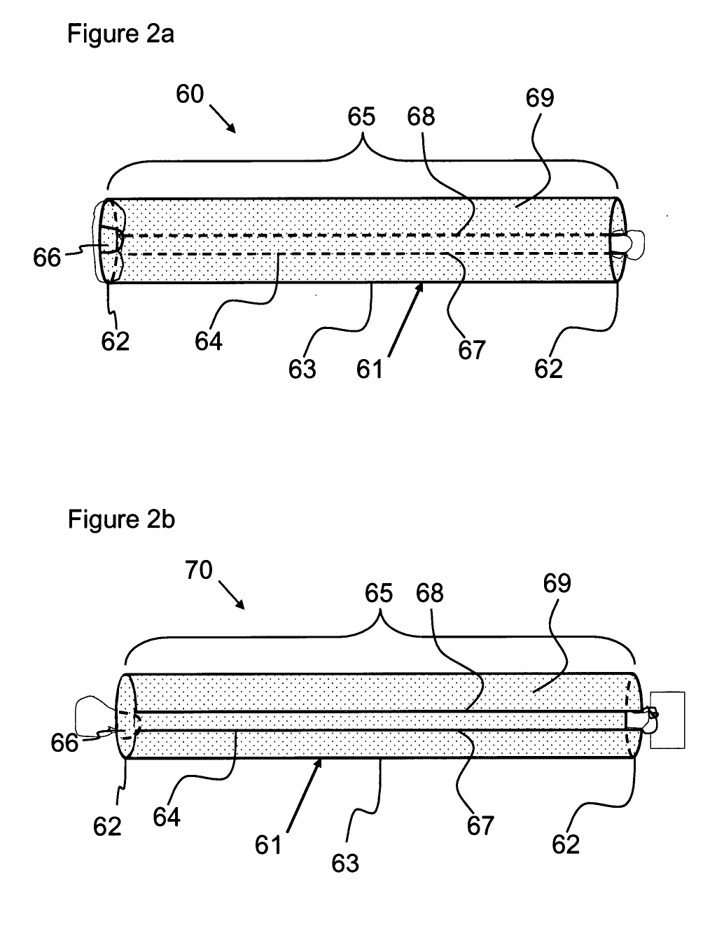

FIGS. 2a and 2b illustrate views of another embodiment of the disposable sanitary cover for shopping device handles wherein the stabilizing means comprises a friction layer: FIG. 2a illustrates a front view shown generally at 60; and FIG. 2b illustrates a back view shown generally at 70. Disposable sanitary cover 60, 70 is appointed to be received on a handle of an array of shopping devices and includes a flexible cylindrical sleeve 61 having first and second ends 62 and a central portion 63. Cylindrical sleeve 61 is composed of a flexible material, preferably a biodegradable coated cardboard or fiber so that disposable sanitary cover 60, 70 is lightweight, easy to use, and environmentally friendly as same is appointed to be disposed of after a single use. A tab member 66 is preferably integrated within cylindrical sleeve 61 so that disposable sanitary cover 60, 70 can be removed from the handle of the shopping cart without the user having to touch the shopping cart handle. That is to say, tab member 66 is simply flipped-up and pulled upward, so that disposable sanitary cover 60, 70 is readily removed from the shopping device's handle with one hand.

Cylindrical sleeve 61 further comprises an elongated slit 64. Slit 64 extends from each of the first and second ends 62 of cylindrical sleeve 61 forming a first rim 67 and a second rim 68 within cylindrical sleeve 61. Disposable sanitary cover 60, 70 further comprises a stabilizing means 65 integrally constructed therein so as to diminish movement of cylindrical sleeve 61 lengthwise and circumferentially on the handle of the shopping device. In this embodiment, stabilization means 65 comprises a friction layer 69 located within central portion 63 and first and second ends 62 of cylindrical sleeve 61. Friction layer 69 is dispersed within the interior of cylindrical sleeve 61 so that movement of the sleeve 61, both lengthwise and circumferentially, along the handle of the shopping device is prevented. This friction layer 69 may be composed of a granular environmentally friendly substance, such as sand, sprayed and adhered to the interior of cylindrical sleeve 61 during manufacturing.

FIGS. 3a and 3b illustrate views of another embodiment of the disposable sanitary cover for shopping device or hospital cart handles, wherein the first rim of the cylindrical sleeve includes a first flap for complete covering of a hospital cart or shopping device's handle. FIG. 3a illustrates a front view shown generally at 40; and FIG. 3b illustrates a back view shown generally at 80. Disposable sanitary cover 40, 80 is appointed to be received on a handle of an array of hospital carts and shopping devices, and includes a flexible cylindrical sleeve 41 having first and second ends 42 and a central portion 43. Cylindrical sleeve 41 is composed of a flexible material, preferably a biodegradable coated cardboard or fiber so that disposable sanitary cover 40, 80 is lightweight, easy to use, and environmentally friendly as same is appointed to be disposed of after a single use.

A tab member 46 is preferably integrated within cylindrical sleeve 41 so that disposable sanitary cover 40, 80 can be removed from the handle of the shopping cart or hospital cart without the user having to touch the shopping cart or hospital cart handle. Cylindrical sleeve 41 further comprises an elongated slit 44. Slit 44 extends from each of the first and second ends 42 of cylindrical sleeve 41 forming a first rim 47 and a second rim 48 within cylindrical sleeve 41. Disposable sanitary cover 40, 80 further comprises a stabilizing means 45 integrally constructed therein so as to diminish movement of cylindrical sleeve 41 lengthwise and circumferentially on the handle of the shopping device or hospital cart. Stabilizing means 45 has tapered portions 49, as is illustrated in FIGS. 1a and 1b discussed hereinabove.

In this embodiment, first rim 47 of cylindrical sleeve 41 further comprises a first flap 50. First flap 50 substantially overlaps second rim 48 of cylindrical sleeve 41, thereby preventing any gaps associated with elongated slit 44 when disposable sanitary cover 40, 80 is placed on a handle of a shopping device or hospital cart. As such, the user's hands are not exposed to any circumferential portion of the handle when shopping. This, in turn, provides maximum prevention, virtually isolating contact between the user's hands and the handle. At the same time, elongated slit 44 is not compromised so that the disposable sanitary cover 40, 80 can readily be snapped or clipped onto the handle of the shopping device or hospital cart with one hand. After clipping onto the handle of the shopping device, first flap 50 is simply folded over to substantially cover second rim 48 and thereby substantially cover the handle of the shopping device and prevent exposure, which tends to take place via elongated slit 44. Preferably, first flap 50 comprises a fastening strip 81 appointed to engage with second rim 48 of cylindrical sleeve 41. Fastening strip 81 is preferably composed of an adhesive strip having a peel-able wax-coated paper so that the peel-able wax-coated paper is peeled or removed exposing the adhesive, and the adhesive is secured against second rim 48 along elongated slit 44. As a result, disposable first flap 50 is secured to second rim 48 and sanitary cover 40, 80 completely encompasses the handle of the shopping device or hospital cart. To facilitate ready one-handed removal of sanitary cover 40, 80 from the hospital cart or shopping device's handle, tab 46 is assembled with an elongated strip 51 which traverses first end 42, central portion 43 and second end 42 of cylindrical sleeve 41. Tab 46 acts in concert with elongated strip 51 when sanitary cover 40, 80 is to be removed from the handle of the shopping device. Elongated strip 51 is appointed to be torn from cylindrical sleeve 41 to form a release gap 52 and disengage disposable sanitary cover 40, 80 from the handle of the shopping device or hospital cart. As a result, disposable sanitary cover 40, 80 can be readily removed from the handle of the shopping device or hospital cart with a single hand and without contact of the user's hands with the shopping device or hospital cart. Second rim 48 of cylindrical sleeve 41 may include a second flap (not shown), similar in nature to first flap 50. This helps to ensure that sanitary cover 40, 80 will have a circumference suitable for encompassing the entire circumference of the shopping device's handle. It substantially prevents prevent gapping, which could in turn expose the user's finger tips to the contaminated handle. In this manner, fastening strip 81 of first flap 50 would be appointed to engage with the optional second flap of second rim 48.

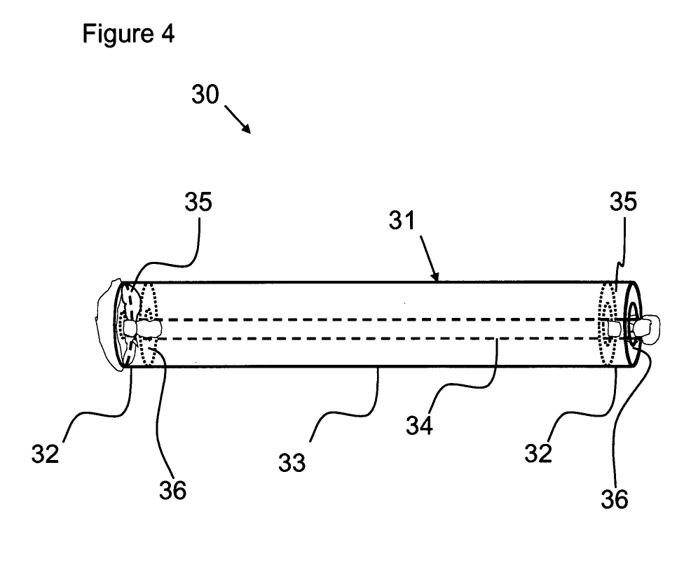

FIG. 4 illustrates a front view of another embodiment of the disposable sanitary cover for temporarily housing a handle of a shopping device or hospital cart wherein the stabilizing means comprises band portions, shown generally at 30. Disposable sanitary cover 30 comprises cylindrical sleeve 31 having first and second ends 32 and a central portion 33. The cylindrical sleeve 31 further comprises an elongated slit 34. Slit 34 extends from each of the first and second ends 12 of cylindrical sleeve 31. A handle of a shopping cart or hospital cart appointed to be pushed by a shopper or a handle of a shopping basket appointed to be carried by a shopper, or a handle of a hospital cart appointed to be pushed by hospital personnel is received through elongated slit 34 so that the handle is housed within the flexible cylindrical sleeve 31 and the passage of germs from the shopping cart's (or basket's) handle to a shopper's hands, or from a hospital cart's handle to the hands of a hospital worker, is mitigated.

The first and second ends 32 of the cylindrical sleeve 31 further comprise a stabilizing means 35 integrally constructed therein. Stabilizing means 35 is shown as band portions 36, wherein the bands 36 cause the first and second ends 32 to have smaller radii than the central portion 33 of the cylindrical sleeve 31. In this manner, first and second ends 32 tightly grasp the handle of the shopping or hospital cart or basket in order to mitigate or prevent rotation of cylindrical sleeve 31, while also mitigating shifting of the cylindrical sleeve 31 from left to right or to and fro. Alternatively, band portions 36 may further comprise a frayed inner portion so that the band portions 36 fit even more snuggly on the handle of the cart or basket. Prevention of the cylindrical sleeve 31 from rotating and/or moving to and from while placed on the handle of the shopping or hospital cart or basket has advantages in that the arrangement mitigates unwanted exposure of the user's hands to handle areas that are sought to be covered. Also, the stabilization means mitigates the risk of the cylindrical sleeve 31 from falling off of the handle of the basket or cart after repeated hand activity of the user.

Having thus described the invention in rather full detail, it will be understood that such detail need not be strictly adhered to, but that additional changes and modifications may suggest themselves to one skilled in the art. For example, disposable sanitary cover 30 can be associated with bars of the type present on hospital beds. In such an embodiment, the first and second ends 32 of the cylindrical sleeve 31 can comprise stabilizing means 35 integrally constructed therein. The stabilizing means 35 would comprise a plurality of band portions 36, wherein the bands 36 would cause the first and second ends 32 to have smaller radii than the central portion 33 of the cylindrical sleeve 31. In this manner first and second ends 32 would tightly grasp the bar of a hospital bed with which the cylindrical sleeve 31 was associated. The cylindrical sleeve 31 could be kept in place on the bar of a hospital bed during a predetermined time corresponding to the time that the bed was occupied by a patient. A patient attempting to rise up to a higher position in the bed, or to stabilize his or her position while standing next to the bed, would find advantageous the presence of the cylindrical sleeve 31 and stabilizing means 35 when grasping the bar. Similarly benefited by the presence of the cylindrical sleeve 31 and stabilizing means 35 on a hospital bed bar are those personnel within the hospital environment that oftentimes move hospital beds from one place to another. In each of these situations the presence of the cylindrical sleeve 31 and stabilizing means 35 provide protection against spread of infectious bacteria and prevent occupants and hospital personnel from accidents occasioned by hospital beds having uncovered or unstable bars. These and other like modifications are intended to fall within the scope of the invention as defined by the subjoined claims.

What is claimed is:

1. A disposable sanitary cover for temporarily housing a handle of a shopping device or hospital cart, and mitigating viral and germ contact on a shopper's or hospital worker's hands, comprising:
a. a cylindrical sleeve having a first end, a second end, and a central portion, and being composed of a flexible material;
b. said cylindrical sleeve further comprising an elongated slit forming a first rim and a second rim within said cylindrical sleeve; and
c. a stabilizing means integrally constructed within said cylindrical sleeve, wherein said stabilizing means mitigates movement of said cylindrical sleeve along said handle of said shopping device or hospital cart;
whereby said handle of said shopping device or hospital cart is received through said elongated slit and is housed within said cylindrical sleeve in order to mitigate said viral and germ contact between said handle of said shopping device or hospital cart and said shopper's or hospital worker's hands.

2. A disposable sanitary cover as recited by claim 1, comprising a tab member for removing said disposable sanitary cover from said handle of said shopping or hospital cart.

3. A disposable sanitary cover as recited by claim 1, wherein said stabilizing means comprises tapered portions located on said first and second ends of said cylindrical sleeve, wherein said tapered portions have smaller radii than said central portion and said first and second ends of said cylindrical sleeve.

4. A disposable sanitary cover as recited by claim 1, wherein said stabilizing means comprises band portions located within said first and second ends of said cylindrical sleeve.

5. A disposable sanitary cover as recited by claim 4, wherein said band portions located within said first and second ends of said cylindrical sleeve further comprise a frayed inner portion so that said band portions fit snuggly on said handle of said shopping device or hospital cart.

6. A disposable sanitary cover as recited by claim 1, wherein said stabilizing means comprises a friction layer located within said central portion, said first end, and said second end of said cylindrical sleeve.

7. A disposable sanitary cover as recited by claim 1, wherein said cylindrical sleeve comprises hand grip indentations located near said first and second ends thereof.

8. A disposable sanitary cover as recited by claim 1, wherein said first rim and a second rim of said cylindrical sleeve overlap one another when said disposable sanitary cover is on said handle of said shopping or hospital cart.

9. A disposable sanitary cover as recited by claim 1, wherein said first rim of said cylindrical sleeve further comprises a first flap.

10. A disposable sanitary cover as recited by claim 9, wherein said cylindrical sleeve further comprises a tab member for removal of said disposable sanitary cover from said handle.

11. A disposable sanitary cover as recited by claim 9, wherein said first flap comprises a fastening strip appointed to engage with said second rim of said cylindrical sleeve.

12. A disposable sanitary cover as recited by claim 11, wherein said cylindrical sleeve further comprises a tab assembly with an elongated strip traversing said first end, a central portion and said second end of said cylindrical sleeve being adapted to be tom from said cylindrical sleeve to form a release gap and to disengage said disposable sanitary cover from said handle.

13. A disposable sanitary cover as recited by claim 9, wherein said second rim of said cylindrical sleeve formed by said elongated slit further comprises a second flap.

14. A disposable sanitary cover as recited by claim 12, wherein said first flap comprises a fastening strip adapted to engage with said second flap of said second rim of said cylindrical sleeve.

15. A disposable sanitary cover as recited by claim 14, wherein said cylindrical sleeve further comprises a tab assembly with an elongated strip traversing said first end, a central portion and said second end of said cylindrical sleeve being adapted to be tom from said cylindrical sleeve to form a release gap and to disengage said disposable sanitary cover from said handle.

16. A disposable sanitary cover as recited by claim 1 comprising a disposable packaging appointed to house said disposable sanitary cover prior to use on said handle.

17. A method of using a disposable sanitary cover for temporarily housing a handle of a shopping device or hospital cart for mitigating viral and germ contact on a shopper's or hospital worker's hands, comprising the steps of:
a. removing a cylindrical sleeve from a dispensing device, said cylindrical sleeve comprising a first end, a second end, a central portion, an elongated slit forming a first rim and a second rim, and a stabilizing means integrally associated therewith, wherein said stabilizing means mitigates movement of said cylindrical sleeve along said handle of said shopping device or hospital cart;
b. aligning said handle of said shopping device with said elongated slit and said first and second rims of said cylindrical sleeve; and
c. pressing said cylindrical sleeve downward so that said handle of said shopping device or hospital cart traverses said first and second rim and said elongated slit and becomes inserted and temporarily housed within said cylindrical sleeve;
wherein said handle of said shopping device or hospital cart is received through said elongated slit and is housed within said cylindrical sleeve in order to mitigate said virus and germ contact between said handle of said shopping device or hospital cart and said shopper's or hospital worker's hands.

18. A method of using a disposable sanitary cover for temporarily housing a handle of a shopping device or hospital cart for mitigating viral and germ contact on a shopper's or hospital worker's hands, comprising the steps of:
a. removing a cylindrical sleeve from a dispensing device, said cylindrical sleeve comprising a first end, a second end, a central portion, an elongated slit forming a first rim and a second rim, and a stabilizing means integrally associated therewith, wherein said stabilizing means mitigates movement of said cylindrical sleeve along said handle of said shopping device or hospital cart;
b. aligning said handle of said shopping device or hospital cart with said elongated slit and said first and second rims of said cylindrical sleeve, said first rim of said cylindrical sleeve further comprising a first flap appointed with a fastening strip and said cylindrical sleeve further comprising a tab assembly with an elongated strip traversing said cylindrical sleeve;
c. pressing said cylindrical sleeve downward so that said handle of said shopping device traverses said first and second rim and said elongated slit and is inserted and temporarily housed within said cylindrical sleeve;
d. folding said first flap of said first rim over said second rim of said cylindrical sleeve and attaching said first flap to said second rim by way of said fastening strip so that said handle of said shopping device or hospital cart is completely temporarily housed within said cylindrical sleeve;
e. using said shopping device for a single shopping outing, after which said cylindrical sleeve is scheduled to be removed from said handle of said shopping or hospital cart;
f. lifting said tab assembly of said cylindrical sleeve and puling said tab assembly and elongated strip so that said elongated strip traverses said cylindrical sleeve, tearing same to thereby form a release gap;
g. removing said cylindrical sleeve from said handle of said shopping device by way of said release gap; and
h. discarding said cylindrical sleeve.

19. A method of using a disposable sanitary cover for temporarily housing a bar of a hospital bed for mitigating viral and germ contact on a shopper's or hospital worker's hands, comprising the steps of:
a. removing a cylindrical sleeve from a dispensing device, said cylindrical sleeve comprising a first end, a second end, a central portion, an elongated slit forming a first rim and a second rim, and a stabilizing means integrally associated therewith, wherein said stabilizing means mitigates movement of said cylindrical sleeve along said bar of said hospital bed;
b. aligning said bar of said hospital bed with said elongated slit and said first and second rims of said cylindrical sleeve, said first rim of said cylindrical sleeve further comprising a first flap appointed with a fastening strip and said cylindrical sleeve further comprising a tab assembly with an elongated strip traversing said cylindrical sleeve;
c. pressing said cylindrical sleeve downward so that said bar of said hospital bed traverses said first and second rim and said elongated slit and is inserted and temporarily housed within said cylindrical sleeve;
d. folding said first flap of said first rim over said second rim of said cylindrical sleeve and attaching said first flap to said second rim by way of said fastening strip so that said bar of said hospital bed is completely temporarily housed within said cylindrical sleeve;
e. using said hospital bed for a time corresponding to a patient's occupancy therein, after which said cylindrical sleeve is scheduled to be removed from said handle of said bar of said hospital bed;
f. lifting said tab assembly of said cylindrical sleeve and puling said tab assembly and elongated strip so that said elongated strip traverses said cylindrical sleeve, tearing same to thereby form a release gap;
g. removing said cylindrical sleeve from said bar of said hospital bed by way of said release gap; and
h. discarding said cylindrical sleeve.
